(19) United States
(12) Patent Application Publication
(10) Pub. No.: US 2009/0216131 A1
(43) Pub. Date: Aug. 27, 2009

(54) USE OF SURFACE MOTION TO IDENTIFY MECHANICAL PROPERTIES OF BIOLOGICAL TISSUE

(76) Inventors: James Geoffrey Chase, Christchurch (NZ); Ashton Peters, North Perth (AU); Thomas Friedhelm Lotz, Christchurch (NZ); Lawrence Allen Ray, Rochester, NY (US)

Correspondence Address: James Geoffrey Chase, 44 Hilltop Drive, Pittsford, NY, 14534, US

(21) Appl. No.: 12/395,205

(22) Filed: Feb. 27, 2009

Related U.S. Application Data
(60) Provisional application No. 61/031,889, filed on Feb. 27, 2008.

(51) Int. Cl. A61B 6/00

(52) U.S. Cl. 600/476

(57) ABSTRACT

A method and apparatus to obtain information about the mechanical properties of a biological tissue by inducing periodic motion in the tissue, imaging the surface motion of the tissue under induced motion, processing the imaged surface motion to obtain descriptive metrics about the surface motion and relating one or more of these descriptive metrics to obtain information about the mechanical properties of the tissue.

### CROSS REFERENCE TO RELATED APPLICATIONS

This application claims the benefit of U.S. Provisional Patent Application No. 61/031,889 filed Feb. 27, 2008, the contents of all of the foregoing applications being incorporated herein by reference in their entirety.

### FIELD OF THE INVENTION

The invention relates generally to the field of mechanical property imaging of biological tissue, and in particular to a technique of breast cancer screening using digital images of the surface motion of the breast under induced motion.

### BACKGROUND OF THE INVENTION

When trying to determine the interior properties of a biological sample, a stimulus or energy is required to be introduced into the sample. A well-known method is x-ray imaging, where the person is exposed to radiation and the body absorbs the radiation differentially depending on the material properties encountered along the ray path. The reduced radiation energies upon exiting the patient are then converted into photons by a phosphorous screen and subsequently captured by a photosensitive media, e.g. film. The patterns on the film are interpreted to deduce the structure and potential pathology of the person. An enhanced x-ray technique is Computer Tomography (CT), in which multiple x-ray images are taken at different angles around the object. A well-known problem with x-rays, computed tomography and other similar radiation based methods, is that the patient is exposed to radiation and there can be long-term health risks with such an exposure.

Another common tissue imaging method is Ultrasound, or more correctly Sonography. Sound waves with frequencies of 2-18 Mhz are focused and emitted into the tissue or body. The sound wave is partially reflected from the layers between different tissues. Specifically, sound is reflected anywhere there are density changes in the body: e.g. blood cells in blood plasma, small structures in organs or stiffer areas within a softer tissue. Some of the reflections return to the transducer and can be used to visualize structures within a tissue or body, including tendons, muscles, joints, vessels and internal organs for possible pathology or lesions. Breast ultrasound is a common imaging method to detect stiffer inclusions within the softer breast tissue, such as cancerous masses. Ultrasound is generally described as a “safe test” because it does not use ionizing radiation, which imposes hazards, such as cancer production and chromosome breakage. However, ultrasonic energy has some potentially hazardous physiological effects, such as enhancing inflammatory response and heating of soft tissue. The imaging performance is also highly dependent on the frequency used and the skill level of the operator.

Magnetic Resonance Imaging (MRI), or nuclear magnetic resonance imaging (NMRI) is a more complex imaging method, which is a medical imaging technique to visualize the structure and function of the body. It provides detailed images of the body in any plane. MRI provides much greater contrast between the different soft tissues of the body than computed tomography (CT) does, making it especially useful in neurological (brain), musculoskeletal, cardiovascular, and oncological (cancer) imaging. Unlike CT, it uses no ionizing radiation, but uses a powerful magnetic field to align the nuclear magnetization of (usually) hydrogen atoms in water in the body. Radiofrequency fields are used to systematically alter the alignment of this magnetization, causing the hydrogen nuclei to produce a rotating magnetic field detectable by the scanner. This signal can be manipulated by additional magnetic fields to build up enough information to construct an image of the body. The benefits or MRI scanning are its detail and contrast, but these are sometimes outweighed by its cost and the duration of the procedure. As the tissue or body is scanned in slices, multiple images need to be taken to obtain a full volume description of the object, which can take up to an hour to obtain.

A problem when comparing different tissue imaging techniques is that different tissue properties are measured. In the case of x-rays, the captured signal is determined by the radiation absorption properties of the materials. Other tissue properties, such as mechanical properties, cannot be measured using x-rays, but have to be measured using alternative methods. One simple method is the palpation technique commonly used by medical doctors to determine the potential for disease—for example, stiffer tissue regions that can be felt as harder objects can indicate the presence of breast or liver malignancies.

There are a number of in-vivo techniques for measuring mechanical properties of tissue. One technique to measure the elastic properties of a biological tissue is called elastography (see Ophir, J. and Céspedes, I. “Method and Apparatus for Elastographic Measurement and Imaging,” U.S. Pat. No. 5,474,070, issued Dec. 12, 1995). In elastographic methods, the tissue to be imaged is stimulated and information about internal properties is determined by analyzing the images of the resultant signal. Elastography typically utilizes a stimulant that is safe to a living being, in that the living being is not exposed to radiation. In most implementations of elastography, the stimulation is in the form of ultrasound. However, in some instance Magnetic Resonance Imaging (MRI) is also used.

Elastography methods can be differentiated between static and dynamic methods. Static Elastography is a medical imaging modality that aims to depict elasticity, a mechanical property of tissue. Elasticity is also referred to as stiffness, or the inverse compliance. The variation of elasticity among tissue types and pathology is well known. Many journal articles describing the clinical applications of elastography are listed by Hall et al. in U.S. Pat. No. 6,508,768. In fact, elastography can be considered as an extension of the traditional diagnostic technique of palpation—the pressing of tissue to feel for differences in elasticity.

The history and development of elastography is given in the following reviews:
J. Ophir, S. K. Alam, B. Garra, F. Kallel, E. Konofagou, T. Krouskop and T. Varghese, “Elastography: ultrasonic estimation and imaging of the elastic properties of tissue”, J. Eng. Med., 213:203-233, 1999.

J. Ophir, I. Cespedes, H. Ponnekanti, and Y. Yazdi, “Elastography: ultrasonic imaging of tissue strain and elastic modulus in vivo”, Eur. J. Ultrasound, 3:49-70, 1996.

L. Gao, K. J. Parker, R. M. Lemer and S. F. Levinson, “Imaging of the elastic properties of tissue—a review”, Ultrason. Med. Biol., 22(8):959-977, 1996.

In static elastography, two images are taken of a region of tissue. One image is taken during compression of the tissue with a nominal static external force. The second image is taken during compression with a larger static external force. The difference between the images is used to calculate relative elasticity. The external force refers to axial pressure applied typically to the surface of the tissue above a region of interest. The basic principle is that stiff tissues will compress less than softer tissues. Dividing each image into small regions and comparing the movement of these regions between the two images provides a quantitative measurement of the local strain. If the stress induced from the external force is uniform throughout the tissue, then estimates of local elasticity can be made. The underlying assumption is that the strain is linearly related to the stress and that this relationship is described mathematically by a linear scale factor called the Young's modulus, or simply “elasticity”. Ultrasound is a common imaging modality for this method. Patents of this approach include those by Ophir et al., in U.S. Pat. Nos. 5,107,837, 5,178,147, 5,293,870, 5,474,070, Konofagou et al. in U.S. Pat. No. 6,270,459, Alam et al. in U.S. Pat. No. 6,514,204, Steinberg et al. in U.S. Pat. No. 5,839,441, Hall et al. in U.S. Pat. No. 6,508,768, Von Behren et al. in U.S. Pat. No. 6,558,324 and Cohen-Bacrie et al. in U.S. Pat. No. 6,176,827. The differences among these patents are mainly in the construction of the apparatus, the methods to compute strain from the ultrasound data and the display of the results.

The problem with these static elastography methods is that they are only able to image a tissue sample under compression and it is usually difficult to compress a larger piece of tissue, especially during in vivo imaging. Even if a larger piece of tissue is imaged in multiple segments, the process would be impractical. Additionally, the tissue properties can only be determined within the compressed tissue, which limits obtainable information on living tissue to regions near the surface of the tissue.

In what we will call dynamic elastography, a force is applied to tissue and the resulting tissue motion is measured, i.e., multiple tissue displacement or velocity measurements are made over a period of time. These measurements can be displayed directly (e.g., magnitude of tissue velocity generated by a vibration source) or following some excitation-dependent signal processing (e.g., the quality factor of the tissue velocity frequency response to a vibration source acting at different frequencies).

Generally, dynamic elastography describes methods that apply mechanical waves globally to a region of tissue using an external vibration source and then measure the resulting tissue motions. The tissue response is usually measured by ultrasound as described in Parker et al. in U.S. Pat. Nos. 5,086,775, 5,099,848, and Lin in U.S. Pat. Nos. 5,919,139, and 6,068,597. Ultrasound is normally used here because Doppler imaging is widely available on commercial ultrasound machines. The Doppler signals measure local velocity within the tissue and the absence of velocity can indicate the presence of stiff inclusions such as tumors.

In the methods disclosed in U.S. Pat. Nos. 5,086,775, 5,099,848, 5,919,139, and 6,068,597, the tissue is excited with a vibrator at audio frequencies and the tissue response is measured by power Doppler measurements. In U.S. Pat. Nos. 5,086,775 and 5,099,848, a mechanical exciter sweeps through a range of audio frequencies until a resonant frequency is detected. In U.S. Pat. No. 5,086,775 Doppler shifted signals are analyzed to find the vibration amplitude of a given region of interest. In U.S. Pat. No. 5,099,848, Doppler shifted signals are analyzed to find the vibration amplitude, the discrete eigenmodes, and eigenfrequency of the tissue. These measurements are then converted into other properties such as shear velocity and Q parameter—the quality factor—and displayed. In U.S. Pat. No. 5,919,139, the Doppler shifted signals are analyzed to find the tissue vibration amplitude, the frequency, and the variance. Various combinations of these properties are displayed. In U.S. Pat. No. 6,068,597, the tissue is vibrated with a wide range of frequencies to obtain the full frequency spectrum of the tissue at different locations. Various measurements of the shape of the spectrum around the resonance peak are then displayed.

In these dynamic elastography approaches using ultrasound, measurements are made of the velocity and resonance characteristics within the tissue. Imaging of larger volumes of tissue require excitation at multiple frequencies for a prolonged period of time, which render it potentially hazardous and complicated for frequent imaging of living individuals, as mentioned before.

Sarvazyan disclosed localized-dynamic-elastography-methods in U.S. Pat. No. 5,606,971, using high-intensity focused ultrasonic waves that are amplitude modulated to generate shear waves at a single location in the tissue. To obtain an image, localized excitations and measurements are repeated at different locations. The shear waves are detected by measuring their amplitude and phase on the surface of the tissue. At least one propagation parameter of the shear waves in the tissue is determined from the phase and amplitude measurements. At least one mechanical parameter of tissue is derived, such as shear elasticity modulus, Young's modulus, dynamic shear viscosity, and mechanical impedance.

The requirement that localized shear waves be used constitutes a significant drawback. Only one small region can be excited at a time and the excitation-measurement process must be repeated for multiple regions, given the rapid spatial decay of shear waves. This reduces the speed of forming a complete image. More importantly, the use of high intensity focused ultrasound in the kHz range poses a possible hazard if the method is used in vivo.

Dynamic elastography methods using acoustic emissions from localized displacements directly measure the acoustic emissions produced by tissue vibrating as a result of a localized oscillating radiation force, as described by Greenleaf et al. in U.S. Pat. Nos. 5,903,516 and 5,991,239. For a constant frequency radiation force, tissues with different viscoelastic properties will produce different emissions. The main idea is to create an oscillating point force in the tissue and measure the emission with a hydrophone. By raster scanning the point source across a region of interest, an image is formed from the magnitude or phase of the measured emissions.

The drawbacks of this approach include the need for specialized equipment for both producing the oscillating point force and measuring the emissions. It also does not measure the underlying properties of the tissue, only the resonance characteristics. Moreover, it requires raster scanning of a region of interest, instead of allowing simultaneous measurements. This reduces the speed of forming a complete image.

As an alternative to ultrasound, elastography imaging can be performed with MRI, as it offers improvements in image quality, but at the expense of speed and cost. Sinkus et al. in U.S. Pat. No. 6,486,669 use a mechanical external excitation and magnetic resonance imaging to extract tissue properties from a linear viscoelastic model. A method is disclosed for vibrating the tissue to create longitudinal mechanical waves with periodic signals, preferably sinusoids, and to obtain the phase and amplitude of the single tone sinusoidal vibrations. To obtain both phase and amplitude, the images and the excitation must be carefully synchronized. Images are taken in slices to obtain a full volume three-dimensional measurement of the object. From these measurements, they solve the wave equation for the viscoelastic model and calculate the model parameters of elasticity, Poison's ratio, tissue density and attenuation. In particular, the time independent solution of the partial differential wave equations is used. With a time independent approach, the tissue must be excited with a periodic signal, such as one or more toned sinusoids, and equilibrium must be reached to eliminate transient responses. Thus, this method is restricted to using excitations with periodic amplitudes to be able to reach equilibrium, where the frequencies are integral multiples.

The requirement that the excitation consist of carefully controlled frequencies and phases in synchronization with magnetic resonance imaging means that a very complicated system is needed compared to other techniques. Another limitation is the need to reach an equilibrium state before measurements can begin. Since tissue relaxation in response to an excitation may take seconds, the reported times required to obtain an image of parameters is of the order of 30 minutes (R. Sinkus, J. Lorenzen, D. Schrader, M. Lorenzen, M. Dargatz, and D. Holz, “High-resolution tensor MR elastography for breast tumour detection”, Phys. Med. Biol. 45, 2000). The vast amount of data obtainable from MRI has its benefits, but also requires large computational expense to solve the mathematical models involved to obtain the mechanical property parameters. These aspects, and the cost involved in MRI scanning, render it highly unpractical for a routine in vivo application.

The material stiffness is the basic mechanical property that is being stimulated and estimated by elastography. Commonly, elastography methods are accompanied by a means to reconstruct the interior of the imaged tissue as a three-dimensional dataset. From such a reconstruction, a stiffness distribution throughout the volume of tissue can be determined, and regions of differential stiffness localized. Such a method is disclosed by Miga et al. in U.S. Pat. No. 7,257,244. Tissue is imaged using MRI or Ultrasound in a pre- and post-deformation state and subsequently the imaging data is used to solve the inverse problem of a mathematical representation of internal mechanical tissue properties. The difficulty with this method is that full volume 3D imaging data is required to solve the mathematical equations. The vast amount of data involved in turn results in highly intensive computational requirements.

Digital Image based Elasto-tomography is an emerging technology for non-invasive breast cancer screening without the requirement of radiation, Ultrasound or MRI (A. Peters, J. G. Chase, EE Van Houten, “Digital image elasto-tomography: combinatorial and hybrid optimization algorithms for shape-based elastic property reconstruction.”, IEEE Trans Biomed Eng. 2008 November; 55(11):2575-83.). As used herein, Digital Image-based Elasto-Tomography system will be referred to as a DIET system. The DIET system uses digital imaging of an actuated breast surface to determine tissue surface motion, which is used to reconstruct the three-dimensional internal tissue stiffness distribution from that motion. Regions of high stiffness suggest cancer since cancerous tissue is between 3 and 10 times stiffer than healthy tissue in the breast. This approach eliminates the need for x-rays and excessive, potentially painful compression of the breast as required in a mammogram.

The DIET system relies on computationally intensive software algorithms to reconstruct the internal elastic property distribution of a biological tissue. This computational effort can take many hours on a supercomputer and is not feasible in a clinical application in which a result is required within minutes. Additionally, due to non-uniqueness issue inherent in these types of optimization problems, an accurate determination of the location and size of the stiffer tissue is difficult to obtain. It thus limits its practical application.

Sinkus et al. discloses in U.S. Pat. No. 7,025,253 a means by which nonlinear disturbances in imaged breast voxels are measured after excitations by mechanical oscillations. Non-linear elastic properties of each voxel are derived from the imaged breast and can be used in addition to normal MRI images to enhance the diagnostic output. Imaging is performed using MRI or Ultrasound techniques. The advantage of this method is that only motion data is used as a diagnostic metric and no computationally expensive inverse problem reconstruction of tissue properties is required. A persistent problem with this method is that imaging of the full volume of the breast is required to obtain three-dimensional information of every voxel within the tissue.

All the imaging methods described here have limitations, either technologically or economically, or both in combination. The goal of all of these methods is to attempt to image the mechanical properties of a biological tissue with the best possible accuracy, trying to obtain a property distribution throughout the full volume of the tissue. This leads to the use of more intense x-rays or ultrasound, longer durations of the imaging procedures, such as seen in MRI, or highly computational software algorithms that take multiple hours to obtain a single diagnostic result. Additionally, specialized costly equipment and highly trained staff are often required. Additionally the equipment is usually large and fixed to a certain location, resulting in a bias in deliverable imaging services between more concentrated urban areas and less populated rural areas.

Therefore, there is a need for a method of:

Imaging the mechanical property characteristics of a biological tissue non-invasively and without radiation;
Imaging the mechanical property characteristics of a biological tissue that does not image the interior of the tissue but only the surface;
Imaging the mechanical property characteristics of a biological tissue that does not require potentially hazardous, high frequency vibrations;
Imaging the mechanical property characteristics of a biological tissue that uses low cost equipment;
Imaging the mechanical property characteristics of a biological tissue that does not require trained radiologist to interpret results;
Imaging the mechanical property characteristics of a biological tissue that can be completed within a short period of time; and
Imaging the mechanical property characteristics of a biological tissue without requiring the solution of highly intense computational algorithms.
Imaging the mechanical property characteristics of a biological tissue with a transportable device.

### SUMMARY OF THE INVENTION

The present invention is directed towards obtaining mechanical property information of a biological tissue by inducing periodic movements in the tissue and imaging the resulting surface motion of the tissue using optical imaging means. Imaging can be performed with still or video cameras, or using other optical measurement methods.

According to one aspect of the invention, motion is induced in the tissue using a mechanical vibration unit. The surface imaging is performed using one or more digital cameras taking multiple timed images during the induced motion of the tissue. These images are processed computationally to obtain the movement of the surface of the tissue in three-dimensional coordinates. The appearance and orientation of the motion pattern on the surface of the tissue gives an indication of mechanical properties within the tissue. One or more descriptive metrics of the surface motion are calculated and used to detect the location of areas with different mechanical properties within the tissue.

One or more combined descriptive metrics derived from the surface motion are compared to a collection of validated metrics obtained from tissue imaged in vivo, in vitro, on phantoms or generated by simulation, to find similar characteristics. These similarities in surface motion are used to obtain descriptive metrics about the mechanical property distribution within the tissue.

In a particular embodiment of the invention, the descriptive motion metrics are compared to descriptive motion metrics obtained from the same tissue during different instances of induced motion to identify changes in tissue properties. This information can be used to track changes in living tissue, such as cancerous tissue developing over time.

Any aspect of a point or region of the surface motion can be used to obtain descriptive metrics, in particular the location, movement and orientation of a point or region on the surface motion of the tissue or the three dimensional surface shape change of the tissue under induced motion.

These and other aspects, objects, features and advantages of the present invention will be more clearly understood and appreciated from a review of the following description of the preferred embodiment and appended claims, and by reference to the accompanying drawings.

### BRIEF DESCRIPTION OF THE DRAWINGS

FIG. 1: A schematic of a possible embodiment of the invention.

FIG. 2: Flowchart showing the procedure to obtain information about the mechanical properties of the tissue.

FIG. 3: Schematic of a possible embodiment of the system setup.

FIG. 4: An example of the images obtained from two different cameras and their combination to calculate the world space positions of the fiducial marker locations.

FIG. 5: A mathematical ellipse matched to the elliptical movement of fiducial markers on the surface of the tissue to create a motion metric defined by the ellipse.

FIG. 6: An example of the difference in surface motion on a healthy tissue and one with a stiffer inclusion.

FIG. 7: An example of imaged surface motion on phantom tissue samples with and without a stiffer inclusion.

FIG. 8: Motion disturbances induced by a stiffer inclusion as seen in the Real (left) and Imaginary (right) parts of the amplitude of the surface motion.

FIG. 9: Difference in motion pattern on the surface of a tissue imaged over a period of time.

FIG. 10: Example of a database that can be used to compare the imaging results to obtain information about the mechanical properties of the tissue.

Corresponding reference characters indicate corresponding parts throughout the several views. The examples set out herein illustrate several embodiments of the invention but should not be construed as limiting the scope of the invention in any manner.

### DETAILED DESCRIPTION OF THE INVENTION

The present invention is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

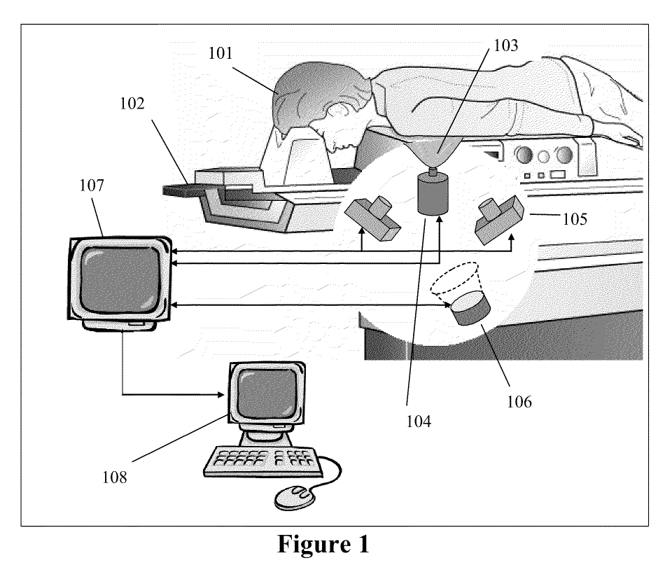

The patient being examined by the present invention may be positioned either standing vertically, or sitting or lying horizontally. One embodiment of the present system is shown in FIG. 1, in which a subject 101 is lying horizontally on a surface 102, with one breast 103 hanging through a hole in the surface 102. The breast 103 is positioned on a vibration unit 104. In the shown embodiment, a single, vertically positioned vibration unit 104 is shown, but the vibration can be applied at any angle and optionally include a plurality of vibration units. The imaging of the surface motion of the breast 103 is performed with at least one camera 105 positioned near the breast 103 and aimed to capture images of the breast 103. However, there are advantages using multiple synchronized cameras 105 where the cameras are positioned in such a manner that each point on the breast 103 is captured in at least two images. The cameras 105 used can be one or any combination of cameras including digital, film, still, video, infrared, laser, or any other optical imaging techniques. It is assumed that all cameras 105 are identical, but this is not a requirement. The system also utilizes a strobe lighting system 106. The strobe lighting system operates at the same frequency as the vibration unit 104, and has the effect to freeze the motion of the breast from the perspective of the camera system 105. The entire system is synchronized by a controller device 107 that controls the vibration units 104, the cameras 105, and the strobe lighting system 106, in such a manner that the vibration units are phased as desired and synchronizing the strobe lighting system 106. While it is assumed that the cameras in the camera system 105 are all triggered simultaneously, it is not a requirement. It is also preferred that the camera system 105, vibration units 104, strobe lighting system 106, and breast 103 are all in a darkened enclosure to ensure the best possible image quality. Images obtained with the camera system 105 are shown, and the image data is subsequently processed with a computational device 108. The computational device 108 can also be programmed in such a manner to perform the function of the controller device 107. In the same manner, the controller device 107 can also be programmed in such a manner to perform the function of the computational device 108.

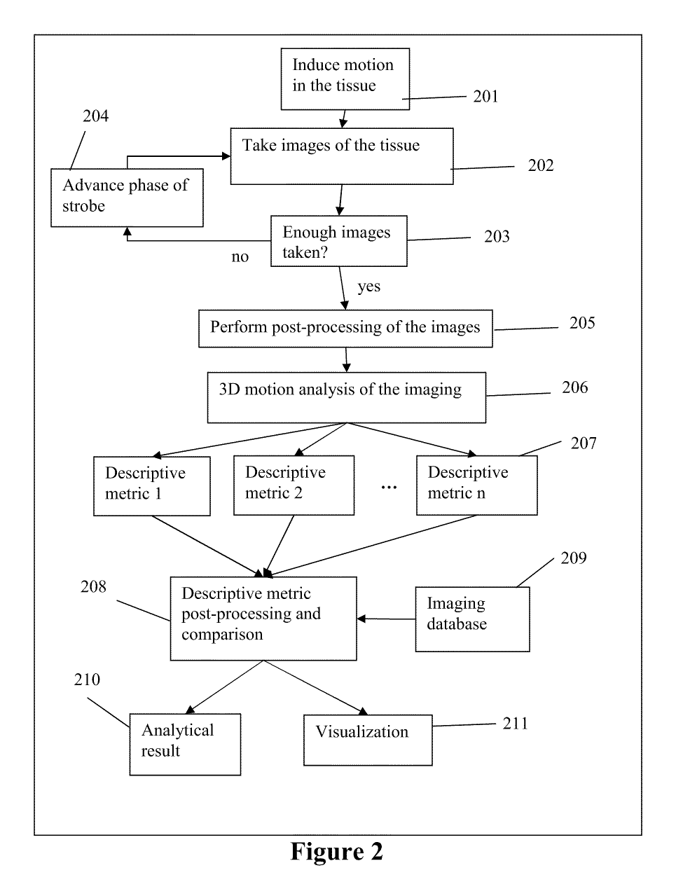

FIG. 2 is a flowchart that details the sequence of steps performed in one embodiment of the present invention. At Step 201 the controller device 107 initiates the vibration unit 104 and sets the strobe lighting system 106 to its first phase. A series of image will be captured, and it will be assumed that N such images, each captured with a different strobe phase offset relative to the frequency of the vibration unit 104. The induced motion provided by the vibration unit 104 is periodic. The frequency of vibration, ω, of unit 104 is variable, and in the range 1-5000 Hz. A typical frequency is about 100 Hz. The amplitude of the vibrating motion is also variable in the range 1 μm-10 mm. The number, size and location of the vibration units 104 can be chosen so as to provide a motion field throughout the tissue volume that reveals the anticipated signal, with clearly defined wave motion at the surface of the tissue.

In step 202 the motion of the breast tissue 103 is captured using the camera system 105. This step has a sequence of three sub-steps that are repeated N times. Step 202 captures one image for each camera in the camera system 105. Step 203 determines if N such captures have occurred. If positive then the process is advanced to Step 205. Otherwise Step 204 is performed and the strobe lighting system 106 is advanced by 2π/ωN seconds. In one embodiment, these sensors are digital still cameras, where the total number of cameras used is enough to capture the whole surface of the tissue. It is preferred that the cameras in the camera system 105 are arrayed in such a manner that every point on the breast 103 can be imaged by at least two cameras. This embodiment has the advantage that motion of the breast tissue induced by the vibration units can be analyzed in three dimensions.

The images taken of the surface of the tissue in step 202 are in a raw format and in the described embodiment of FIG. 1, in two dimensions, meaning that no information about the location in space of the imaged surface is known. These images need to be processed in step 205, to obtain preferably three-dimensional descriptions of the surface motion of the tissue. Processing can be done in a processing unit attached to the system 108, or at a different location and time on any processing unit, such as a personal computer, PDA or dedicated digital or analog processing unit. Following the imaging of the three-dimensional movement of the surface of the tissue in step 205, the motion is analyzed and described using three dimensional motion characteristics in step 206. An example of such a motion characteristic is the ellipticity of the sampled point under induced motion.

The next step in the process is the derivation of descriptive surface motion metrics, step 207. The pattern of surface motion that can be seen on a vibrating tissue sample with homogeneous mechanical properties differs significantly to the pattern seen on a tissue with inhomogeneous mechanical properties. As an example, the presence of a high stiffness inclusion within a tissue with lesser stiffness will cause a disruption to the motion field due to the mechanical wavelength changing within areas of stiffer tissue. It is possible to relate the observed location of motion disturbances to the likelihood of an inhomogeneity at a location within the tissue in the immediate vicinity, or further afield.

The measurable surface motion of a point or region on the surface of the tissue is quantifiable in one or more independent or inter-dependent metrics as shown in step 207. The metrics can include, but are not limited to:

Magnitude of the displacement amplitude.
Real part of the displacement amplitude.
Imaginary part of the displacement amplitude.
Orientation of the displacement amplitude.
Change in Real part of the displacement amplitude between homogenous regions and inhomogeneous regions.
Change in Imaginary part of the displacement amplitude between homogenous regions and inhomogeneous regions.
Orientation of a point or region of the surface under induced motion
Change in orientation of a point or region of the surface under induced motion between homogenous and inhomogenous regions.
Velocity of the marker movement
Change in velocity of the marker movement
Shape of the surface of the tissue
Changes in shape of the surface of the tissue between instances of induced motion on the same tissue
Differences or discontinuities, both absolute or relative, of motion in a region of the surface compared to another region of the surface
Difference in any motion descriptive metrics mentioned above between two instances of induced motion

In step 208 the descriptive metrics obtained in step 207 can be computed in a processing unit 108 to obtain more detailed descriptions of characteristics of the tissue. This post-processing can include one or more computational steps to enhance the signal from the descriptive metrics of step 207. One possible computational step is to combine the signal of the metrics with different weightings to maximize the output signal. Another possible computational step is to characterize patterns derived from the descriptive metrics of step 207, and compare these patterns to descriptive metrics from a database of imaging data 209, populated with one or more tissue imaging results obtained in other instances of induced tissue motion. The metrics in the database can be obtained from in vivo tissue experiments, in vitro tissue experiments, in vitro phantom experiments, or computer simulations. Preferably, the metrics in the database are validated with other imaging techniques, to include further confidence in the strength of the metrics.

The post-processed descriptive metrics are refined in such way in step 208, that an analytical result can be obtained from them in step 210. This analytical result can include crude information about the elastic property distribution within the tissue, or more detailed information about the exact location and characteristics of the tissue with different elastic properties and can be further used independently or in combination with other imaging techniques. Additionally, in step 211 the obtained outcome of step 208 can be visualized graphically on a system such as 108 or similar. This visualization step 211 can include a graphical representation of the obtained metrics in step 208 to enhance the quality of information delivered to the operator.

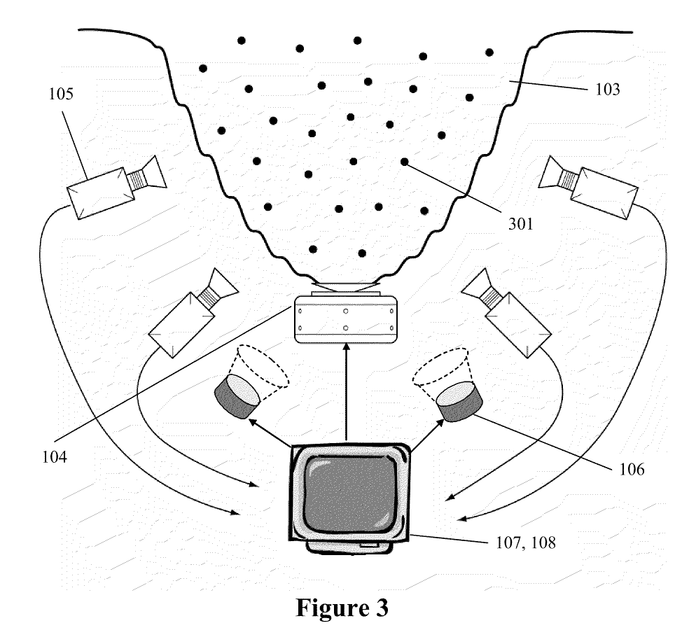

FIG. 3 further describes one embodiment of the process steps indicated by steps 201 and 202 of FIG. 2. In this embodiment, the tissue to be imaged is a human breast 103. The surface of the breast 103 has fiducial markers 301 applied that can be tracked by the imaging system. These fiducial markers 301 can be applied with paint, markers or any material attached to the surface, such as small elements of a material attached with glue. The fiducial markers should be as small as possible, which is dependent upon the resolution of the camera system 105. There should be sufficient fiducial density to assure that all regions on the breast 103 are examined, but not so dense that finding corresponding points in multiple camera frames is impractical. Motion is induced in the tissue using the vibration unit 104. The surface of the breast 103 is imaged using a camera system 105, in this embodiment consisting of four digital cameras arrayed around the breast 103, however, an alternative number of cameras may be used. The digital still cameras 105 are directed by a controller device 107 unit to ensure synchronization with the vibration unit 104. The images obtained from the camera system 105 are transferred to the processing unit 108, which can also be separate to the control unit. Raw imaging data is now available for post processing.

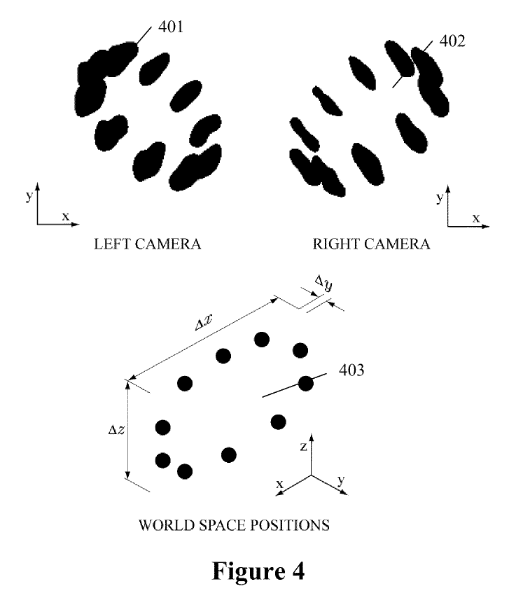

The motion at the surface of the breast 103 can be quantitatively assessed by tracking fiducial markers 301 arrayed over the surface of the breast 103, as shown in the embodiment in FIG. 3. These markers can be either externally applied to the skin prior to imaging, or generated from natural variations in skin tone by an analysis of the captured image sequences. In one embodiment, where more than one camera is used, the three-dimensional position of each fiducial marker can be determined by combining the marker's two-dimensional position information from multiple independent images of the same marker. This process is shown for an example fiducial marker tracked by two cameras in FIG. 4. Element 401 in FIG. 4 shows the combination of all images obtained by the left camera, and element 402 the images obtained by the right camera, in a two-camera imaging setup. Each one of the black dots is the image of the fiducial marker at a defined point in time during the vibration of the tissue. The movement of the fiducial markers 301 can be seen by the elliptical pattern the marker follows. The same markers are tracked by both cameras at a different angle. Using commonly known methods to those skilled in the art, such as a triangulation algorithm, the two camera images can be combined to form a three-dimensional movement image 403 of every fiducial marker imaged on the surface of the tissue

In another embodiment of the invention, surface motion of the tissue can also be derived by tracking natural patterns on the surface of the skin using methods such as Normalized Cross-Correlation (NCC) [J. P. Lewis (1995), “Fast Normalized Cross-Correlation”, Vision Interface, 120-123] or any other feature or intensity based methods such as, but not limited to, those published in the literature [Jan Modersitzki (2004), “Numerical Methods for Image Registration”, Oxford University Press, New York].

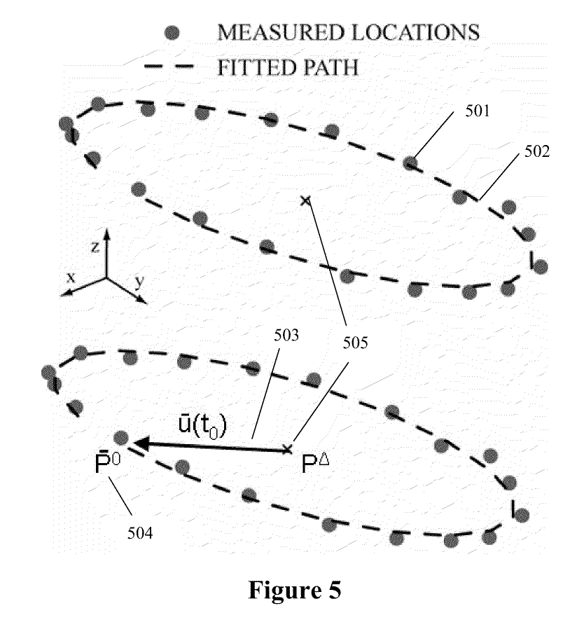

FIG. 5 shows an example of an embodiment of the motion characterization performed in step 206 of FIG. 2. The motion of the fiducial markers 501 are approximated by a theoretical motion path described in three dimensions using parametric mathematical equations, in this case with the shape of an ellipse 502. A possible mathematical description of the elliptical motion pattern using a complex description is shown in more detail herein. A plurality of other mathematical descriptions can be used to describe the same elliptical motion pattern.

The steady state oscillation of a point about an origin can be expressed using a time-harmonic displacement vector 503, defined at any time t as

ū(t)= [symbol] {ue^{iwt},} (1)

where ω is the frequency of the system, u the magnitude of the amplitude, and the amplitude term ū(t) contains both real and imaginary components u_{R} and u_{I}:

u=u_{R}−iu_{I} (2)

The measured position of a reference point at time t₁, P̅ⁱ (exemplary point P̅⁰ shown at 504), can be approximated as the addition of a harmonic displacement, ū(tᵢ), to the reference point centroid, P̅^{Δ} (504), as

P̅ⁱ=ū(tᵢ)+P^{Δ} (3)

Alternatively, the reference point motion can be described using a real-valued amplitude, ũ, and a phase, φ, yielding

ū(t)= [symbol] {ũe^{i(wt+φ)}} (4)

The motion description in Equation (4) can be converted to the form of Equation (1) by relating the real-valued damped amplitude, ũ, and phase, φ, to a complex amplitude, u=u_{R}−iu_{I}, as follows:

[symbol]

{ue^{iwt}}= [symbol] {ũe^{i(wt+φ)}} (5)

[symbol]

{ue^{iwt}}= [symbol] {ũe^{iwt}e^{iφ}} (6)

u=e^{iφ} (7)

u_{R}−iu_{I}=ũ cos(φ)−iũ sin(φ) (8)

Both of these motion components, real and imaginary, can contain valuable information to describe the motion on the surface of the tissue. They can be used independently or combined with others to provide one or more descriptive metrics of the motion. The identification of the parameters involved can be performed using an error optimization approach, where the error between a theoretical marker path and the measured path is minimized.

Yet another possible mathematical description of an ellipse is shown here. The motion of a fiducial marker is modeled as an ellipse and may be assumed to be contained within a plane. An ellipse is described by the major and minor axes, and by taking the ratio of the length of these axes determines a measure of the orbit's ellipticity. A model of the fiducial marker's orbit is:

Orbit(t)=(α₁+β₁ sin(2πω(t+φ₁)),α₂+β₂ sin(2πω(t+φ₂))) (9)

where (α₁, α₂) is the center of the orbit; ω is the actuator frequency; β₁ and β₂ are the extent of the ellipse; and ω₁, and ω₂ are the phase of the orbit.

For the present purposes the model of the orbit can be simplified by assuming the orbit's center is at the origin, the extent is a relative, and the phase offsets are relative. The simplified model is:

Orbit(t)=(sin(t), β sin(t+φ)) (10)

The major and minor axes intersect the orbit at the minimum and maximum distances from the center (origin). If the orbit is on the major axis at time to, then it is on the minor axis at times t₀+π/2 and t₀−π/2. The squared distance of the orbit from the origin is simply:

D(t)=sin²(t)+β² sin²(t+φ) (11)

Using standard methods from elementary calculus, an extreme point of the function is found at time

$${t}_{0} = \frac{1}{2} {tan}^{- 1} ( \frac{sin ( ϕ ) cos ( ϕ )}{1 + {β}^{2} cos ( 2 ϕ )} ) \qquad ( 12 )$$

The ellipticity metric EM for the orbit is

$$EM = \sqrt{\frac{D ( {t}_{0} )}{D ( {t}_{0} + π / 2 )}} \qquad ( 13 )$$

provided the number is greater than or equal to unity and the reciprocal otherwise. The measure of ellipticity EM of the motion ellipse 502 can be an indicator of a motion descriptive metric 207 to describe inconsistencies in surface motion. One possible mathematical description is shown here, but a plurality of other methods can be used to describe the same characteristics.

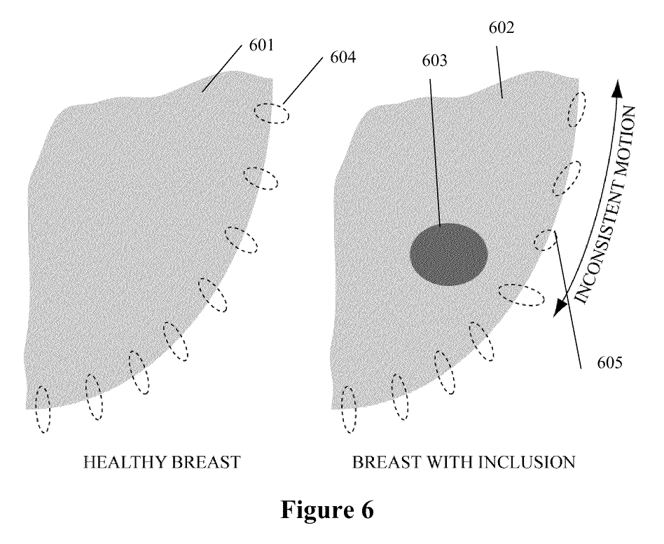

An example of the disturbance of the surface motion as a result of a stiffer inclusion in a lesser stiff tissue is shown in FIG. 6. FIG. 6 shows one embodiment of the invention, in which the imaged tissue can be a human breast 103. The breast shown on the left 601 is a healthy phantom breast with softer homogenous tissue distribution throughout its volume, and the breast on the right 602 is a phantom breast with a stiffer inclusion 603, i.e. a cancerous lesion. The surface motion of the fiducial markers on the breast 604-605 visualizes clearly the significant differences seen in a healthy breast 601 and in one with a stiffer cancerous inclusion 602. The inconsistency in motion pattern seen in 605 is indicative of the stiffer inclusion 603. Note that the elliptical movements change their shape and orientation, both normal and perpendicular to the surface of the breast.

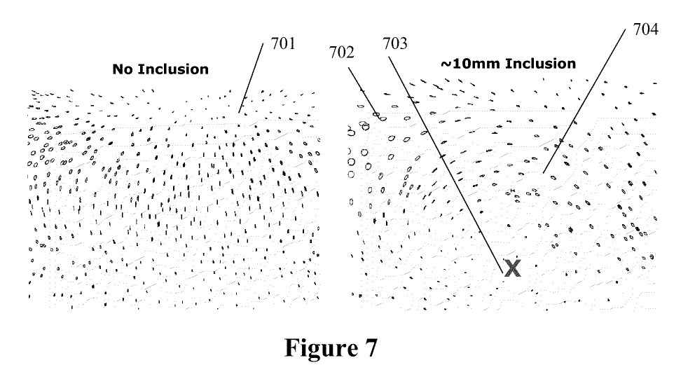

Another example of this disturbance can be seen in FIG. 7, in which the surface motion of a silicone phantom breast with no inclusion 701 is compared to the surface motion of a silicone phantom breast with a 10 mm inclusion 702. The pattern of the fiducial markers around the area of the inclusion 703 is clearly different to 701. The presence of an inclusion within the breast 103 causes a measurable change in the displacement field at the surface in the vicinity of the inclusion. Motion field disruption in this case can be described using descriptive motion metrics in the form of phase and/or amplitude variations in each of the three orthogonal directions, during either steady-state or transient motion.

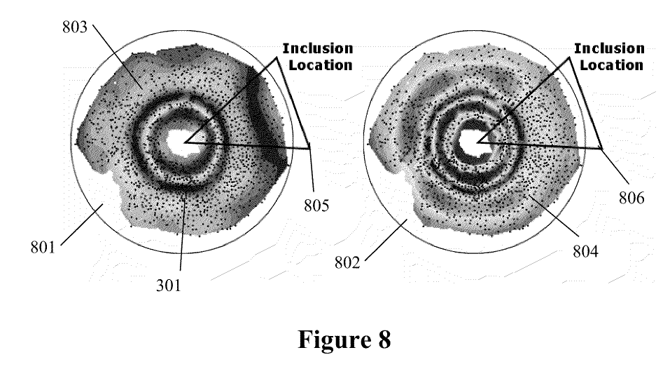

FIG. 8 shows the surface motion of a silicone phantom breast in a projected view perpendicular to the vibration source. Both images 801 and 802 show different components of the amplitude of the surface motion. The dots are tracked fiducial markers 301. Image 801 shows the real component of the amplitude of motion, and image 802 shows the imaginary component of the amplitude of motion. The intensity of the amplitude metrics is visualized by different shades of grey 803-804, with the intensity increasing from lighter to darker shades. The area of the location of the stiffer inclusion is indicated by the triangles 805 and 806. The disturbances in the surface motion pattern are clearly evident in 801 by the larger dark section within the triangle 805 at the outskirts of the image 801. Less clear but also evident is the disturbance within triangle 806 on image 802. Depending on the size and location of the stiffer inclusion, and on the induced motion settings, real and imaginary parts of the motion amplitude can show differently strong signals. One or more of these signals can be used independently or combined to provide information about the mechanical properties of the tissue.

A further example of a descriptive metric 207 is the change in any other descriptive metric 207 when successive motion fields from a single tissue sample are compared over time to detect changes in tissue properties. A gradual or sudden change in the observed motion field can be caused by a change in the internal stiffness distribution within the tissue, therefore quantitative and qualitative analysis of the motion field variation can provide evidence as to the presence of internal areas with higher stiffness.

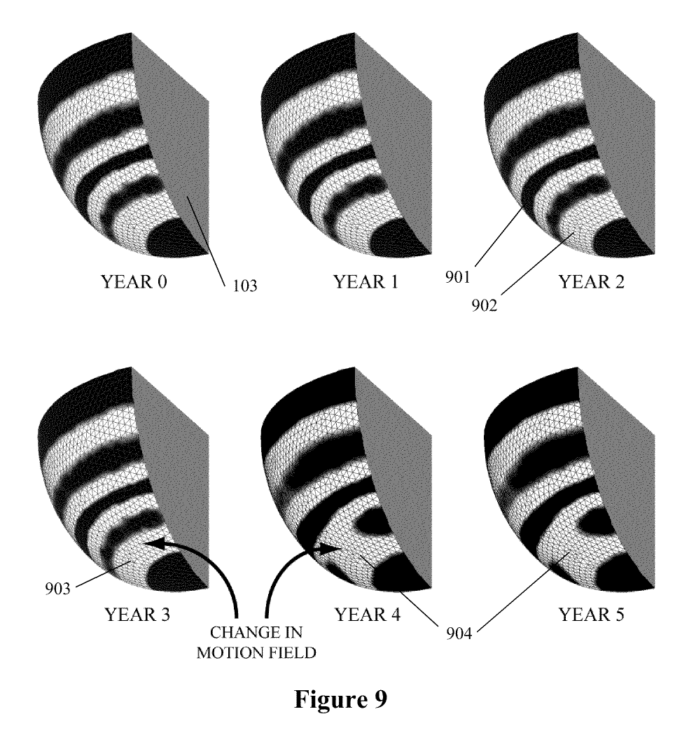

A particular embodiment of this metric is shown in FIG. 9. A breast 103 is imaged over a period of time, in this example over 5 years. Every image shows a motion field defined by darker colored peaks 901 and lighter colored troughs 902. Images taken between years 0 to 3 show similar motion patterns. Descriptive metrics are very similar every year and if the difference is computed, no change in tissue properties can be identified. On the image taken at year 4, a change is visible as seen in the lower area 904, compared to the previous year 903. This disturbance in the surface motion is an indication of a change in tissue properties between year 3 and 4. The descriptive metric computed from the year 4 image can be used independently, but a stronger signal is obtained if the change in surface motion is included in the analysis. The change in surface motion from 903 to 904 can also be used independently to obtain an analysis of a change in tissue properties, for example the appearance of a stiffer cancerous inclusion. In a further embodiment, this change in surface motion can be used to build a history of a particular person's tissue properties to better monitor physiological changes and detect malignant changes in a more timely manner.

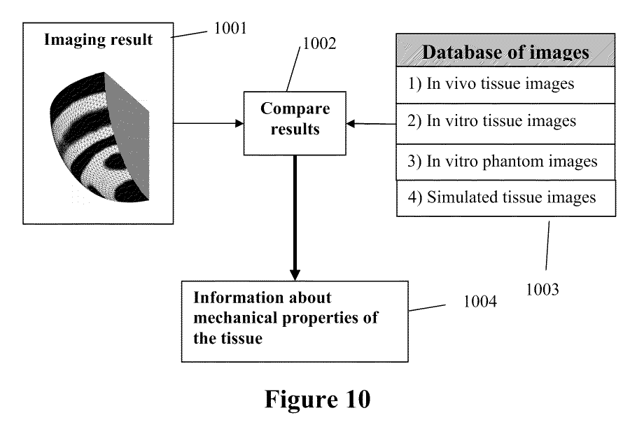

FIG. 10 shows in more detail step 209 in FIG. 2, the process 1002 used to compare an imaging result 1001 to previously obtained imaging data in a database 1003. The imaging result 1001 or any single or combination of descriptive metrics obtained in step 207 can be compared to the results obtained from one or more other images in a database 1003. The database can be populated with images obtained from a variety of sources. They can be imaged in vivo on live tissue, in vitro on diseased tissue, in vitro on phantom tissue constructed synthetically from other material with similar mechanical properties, simulated with a computer model in a processing unit, or using any other means to obtain images similar to the tissue of interest. The images in the database 1003 are preferably validated on other imaging modalities such as x-ray, MRI, CT or ultrasound, to increase their validity. Pattern characterization algorithms can also be used to obtain similarity metrics between the imaging result 1001 and the database 1003. The imaging result 1001 can then be compared 1002 to the database 1003 to find similarities that can provide information about the mechanical properties of the tissue 1004. This information 1004 can be used independently or combined with one or more descriptive metrics 207 in step 208 to improve the final analysis 210.

What is claimed is:

1) A method of obtaining information about mechanical properties of tissue by analyzing the surface motion of said tissue during induced periodic motion, said method comprising the steps of:
a) Inducing periodic motion in the tissue;
b) Imaging the surface motion of the tissue under induced motion;
c) Processing the imaged surface motion to obtain descriptive metrics about said surface motion;
d) Relating one or more of these descriptive metrics to obtain information about the mechanical properties of said tissue.

2) The method of claim 1, wherein the periodic motion is induced mechanically or sonically.

3) The method of claim 1, wherein the periodic motion is induced by one or more vibration units.

4) The method of claim 1, wherein the periodic motion is a sinusoidal oscillation of one or more periods.

5) The method of claim 1, wherein the motion tracking is done with at least one digital still camera.

6) The method of claim 1, wherein the motion tracking is done with at least one digital video camera.

7) The method of claim 1, wherein the motions are tracked using externally applied fiducial markers on the surface of the tissue.

8) The method of claim 1, wherein the motions are tracked using natural tissue patterns on the surface of the tissue.

9) The method of claim 1, wherein the descriptive metrics processed are amplitude and phase of a point or region of the surface under induced motion.

10) The method of claim 1, wherein the descriptive metrics processed are ellipticity of a point or region of the surface under induced motion.

11) The method of claim 1, wherein the descriptive metrics processed are the change of shape of a region of the surface under induced motion.

12) The method of claim 1, wherein the descriptive metrics processed are the direction of motion of a point or region of the surface under induced motion.

13) The method of claim 1, wherein the descriptive metrics processed are differences or discontinuities, both absolute or relative, of motion in a region of the tissue surface compared to another area of the tissue surface.

14) The method of claim 1, wherein the descriptive metrics processed are the differences in surface motion between two or more instances of induced motion on the same tissue.

15) The method of claim 1, wherein the descriptive metrics are compared to one or more instances of induced motion obtained from in vivo studies, in vitro studies, phantom tissue studies or simulations.

16) The method of claim 1, wherein the mechanical properties of said tissue are elastic properties.

17) The method of claim 1, wherein the analyzed tissue is a human breast.

18) The method of claim 16, wherein the elastic properties relate to a cancerous or benign lesion within a human breast.

19) The method of claim 1, wherein the information about mechanical properties of said tissue is visualized graphically.

20) A system and apparatus to obtain information about the mechanical properties of tissue by analyzing surface motion of said tissue during induced periodic motion, comprising the means to:
a) Induce periodic motion in the tissue;
b) Image the surface motion of the tissue under induced motion;
c) Process the imaged surface motion to obtain descriptive metrics about said surface motion;
d) Relate one or more of these descriptive metrics to obtain information about the mechanical properties of said tissue;
e) Visualize graphically said information about the mechanical properties of said tissue.
